(19) United States
(12) Patent Application Publication
(10) Pub. No.: US 2018/0244615 A1
(43) Pub. Date: Aug. 30, 2018

(54) USE OF COMPOUNDS INCLUDING A SULFOXIDE OR SULFONE FUNCTION AND AN AMIDE FUNCTION AS SOLVENTS AND NEW SOLVENTS

(71) Applicant: ARKEMA FRANCE, Colombes (FR)

(72) Inventors: Paul Guillaume SCHMITT, Lescar (FR); Jean-Alex Laffitte, Pau (FR)

(73) Assignee: ARKAMA FRANCE, Colombes (FR)

(21) Appl. No.: 15/754,712

(22) PCT Filed: Sep. 15, 2016
(86) PCT No.: PCT/FR2016/000137

§ 371 Date: Feb. 23, 2018

(30) Foreign Application Priority Data
Sep. 15, 2015 (FR) 1558601

(51) Int. Cl. C07D 207/27; C07C 317/28; C09D 7/20; C11D 3/43; C11D 3/34; C11D 7/50; C10M 105/72; A01N 25/02; A61K 47/20; A61K 47/22

(52) U.S. Cl. CPC C07D 207/27; C07C 317/28; C09D 7/20; C11D 3/43; C11D 3/349; C11D 3/3445; C10M 2219/09; C10M 105/72; A01N 25/02; A61K 47/20; A61K 47/22; C10M 2219/0406; C11D 7/5009

(57) ABSTRACT

Provided is a composition that contains a compound a) and at least one chemical entity b) dissolved in compound a). Compound a) has at least one sulfoxide functional group and/or one sulfone functional group, and at least one amide functional group. The composition is useful for a variety of applications, including manufacturing a film, a coating on a support, a hollow fiber, an artificial leather, a polymeric fiber, a membrane, a separator or an electrode for batteries, an electronic circuit or a sheath for the protection of electric cables.

### FIELD OF THE INVENTION

The invention relates to the technical field of the solvents used in particular to dissolve a polymer or to dissolve an active entity exhibiting pharmaceutical or plant protection properties.

### TECHNICAL BACKGROUND

Polymer solutions can be obtained by dissolving a polymer in one or more organic solvents, such as N-methylpyrrolidone (NMP), dimethylacetamide (DMAc) or dimethylformamide (DMF).

After removal of the solvent or solvents, for example by evaporation or by extraction using a third solvent, or any other method known to a person skilled in the art, a polymer film or a hollow polymer fiber can be obtained. Such films or fibers have numerous applications, such as the coating of textiles, in particular artificial leather; separators or electrodes for batteries; membranes for the treatment of water or dialysis; protection of electric cables by sheathing and electronic circuits.

The abovementioned solvents are nevertheless regarded as mutagenic and reprotoxic (“CMR” compounds). Their use thus presents a risk to the health of the user. Consequently, novel solvents which can replace them have been looked for.

The document U.S. Pat. No. 8,735,324 provides for the use of an ester amide in order to dissolve plastics.

The documents WO 2013/155659 and WO 2014/001100 provide for the use of a solvent comprising a mixture of an ester amide and of dimethyl sulfoxide (DMSO) in order to dissolve a fluoropolymer.

The document WO 2014/096071 provides for the use of an ester amide, generally as a mixture with DMSO, in order to dissolve a sulfonated polymer.

In the last three documents cited, the ester amide is generally used as a mixture with DMSO. In point of fact, the use of two cosolvents renders the implementation of the dissolution process complex on the industrial scale. The recycling process is also complex as it requires managing the recycling of two cosolvents. Furthermore, depending on the temperature and pressure conditions, and also on the type of polymer to be dissolved, phenomena of phase separation of the two cosolvents, that is to say the separation into two phases each consisting of one of the two cosolvents, may be observed.

The document WO 2013/107822 provides for the partial or complete replacement of the toxic solvents, such as NMP, DMF or DMAC, by a solvent chosen from N-butylpyrrolidone, N-isobutylpyrrolidone, N-(t-butyl)pyrrolidone, N-(n-pentyl)pyrrolidone, N-((methyl-substituted)butyl)pyrrolidone, N-propyl- or N-butylpyrrolidone, the ring of which is methyl-substituted, or N-(methoxypropyl)pyrrolidone, it optionally being possible for these solvents to be mixed with DMSO.

A search is thus underway to replace the mixture of a molecule having an amide functional group and of DMSO with just one solvent.

### SUMMARY OF THE INVENTION

To this end, the invention provides for the use, as solvent, of a compound comprising:
a sulfoxide —S(═O)— functional group and/or a sulfone —S(═O)₂— functional group and
an amide functional group.

According to a preferred embodiment, the composition comprises:
a compound a) comprising:
   at least one sulfoxide functional group and/or one sulfone functional group, and
   at least one amide functional group, and
at least one chemical entity b) dissolved in the compound a), said compound a) corresponding to the formula (I):

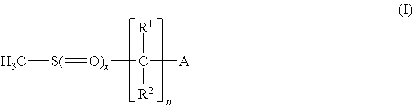

in which:
x is equal to 1 or 2;
R¹ and R² represent, independently of one another, a hydrogen atom or an alkyl group comprising from 1 to 6 carbon atoms;
n ranges from 1 to 10;
A represents one of the following groups:

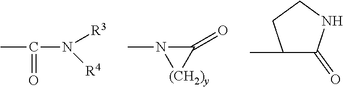

where:
if x=1, then R³ and R⁴ represent, independently of one another, a hydrogen atom or an alkyl group comprising from 1 to 20 carbon atoms;
if x=2, then R³ and R⁴ represent, independently of one another, an alkyl group comprising from 1 to 20 carbon atoms; and
y ranges from 1 to 10.

The term “solvent” is used in the present patent application in its normal sense, that is to say that it denotes a substance capable of dissolving another substance (solute) without chemically modifying it and without itself being modified. In the case where the solute is a polymer, the term of solution of polymer in a solvent is used when the mixture obtained is homogeneous.

The term “polymer”, within the meaning of the present invention, denotes any molecule exhibiting at least two identical units (monomers) connected via a covalent bond.

This compound exhibits the distinguishing feature of combining, in the same molecule, a sulfoxide and/or sulfone functional group and an amide functional group.

According to one embodiment, the compound corresponds to the formula (I):

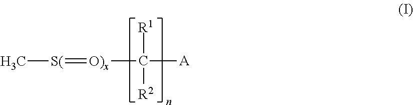

in which:
x is equal to 1 or 2;
R¹ and R² represent, independently of one another, a hydrogen atom or an alkyl group comprising from 1 to 6 carbon atoms;
n ranges from 1 to 10, preferably from 1 to 6 and more preferably from 1 to 2;
A represents one of the following groups:

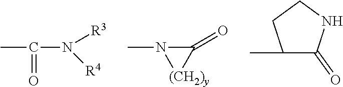

where:
R³ and R⁴ represent, independently of one another, a hydrogen atom or an alkyl group comprising from 1 to 20 carbon atoms;
y ranges from 1 to 10, preferably from 2 to 4 and is preferably equal to 3.

According to one embodiment, R¹ and R² each represent a hydrogen atom.

According to one embodiment, R³ represents a hydrogen atom and R⁴ represents an alkyl group comprising from 1 to 20 carbon atoms.

According to one embodiment, R³ and R⁴ each represent an alkyl group comprising from 1 to 20 carbon atoms, preferably chosen from the methyl and ethyl groups.

According to one embodiment, R³ and R⁴ each represent a methyl group.

Preferred compounds have the formula:

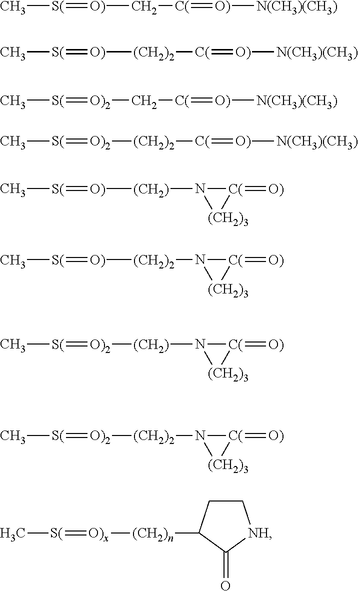

with x=1 and n=2.

According to one embodiment, the composition comprises:
a compound a) comprising:
   at least one sulfoxide functional group and/or one sulfone functional group, and
   at least one amide functional group, and
at least one chemical entity b) dissolved in the compound a).

According to a preferred embodiment, the composition comprises:
a compound a) comprising:
   at least one sulfoxide functional group and/or one sulfone functional group, and
   at least one amide functional group, and
at least one chemical entity b) dissolved in the compound a), said compound a) corresponding to the formula (I):

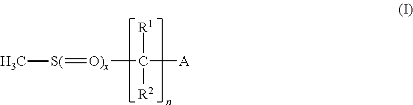

in which:
x is equal to 1 or 2;
R¹ and R² represent, independently of one another, a hydrogen atom or an alkyl group comprising from 1 to 6 carbon atoms;
n ranges from 1 to 10;
A represents one of the following groups:

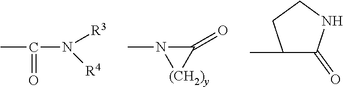

where:
if x=1, then R³ and R⁴ represent, independently of one another, a hydrogen atom or an alkyl group comprising from 1 to 20 carbon atoms;
if x=2, then R³ and R⁴ represent, independently of one another, an alkyl group comprising from 1 to 20 carbon atoms; and
y ranges from 1 to 10.

According to one embodiment, the compound a) is present at at least 45% by weight in the composition, preferably from 45% to 99.9% by weight, more preferably from 50% to 99% by weight, the limits being included.

In the composition according to the present invention, the at least one chemical entity b) dissolved in the compound a) is a polymer. Thus, the compound according to the invention is advantageously used as solvent for dissolving polymers but also as solvent for dissolving chemical entities active in the pharmaceutical or plant protection field.

According to one embodiment, the composition additionally comprises another solvent, which represents less than 90% by weight, with respect to the total weight of the composition.

According to one embodiment, the other solvent is chosen from water, ketones, amines, alcohols, ethers, esters, sulfones, aromatic compounds or acetals or from N-butylpyrrolidone, N-isobutylpyrrolidone, N-(t-butyl)pyrrolidone, N-(n-pentyl)pyrrolidone, N-((methyl-substituted)butyl)pyrrolidone, N-propyl- or N-butylpyrrolidone, the ring of which is methyl-substituted, or N-(methoxypropyl)pyrrolidone, dipropylene glycol dimethyl ether (DPGDME), polyglyme, ethyl diglyme, 1,3-dioxolane or methyl 5-(dimethylamino)-2-methyl-5-oxopentanoate.

According to one embodiment, the composition additionally comprises dimethyl sulfoxide (DMSO).

According to one embodiment, the polymer is chosen from the group consisting of polyvinylidene fluoride (PVDF), polytetrafluoroethylene (PTFE), a polyurethane (PU), a polyimide (PI), a polyester-imide (PEI) and a polyamide-imide (PAI) and sulfonated polymers, such as polyethersulfones (PES), polysulfones (PSU) and polyphenylsulfones (PPSU), preferably chosen from the group consisting of polyvinylidene fluoride (PVDF), polyurethane (PU), polyethersulfones (PES), polysulfones (PSU) and polyphenylsulfones (PPSU).

According to one embodiment, the composition is used as plant protection composition, pharmaceutical composition, stripping composition, degreasing composition, cleaning composition, lubricating composition, coating composition or pigment composition.

Another subject matter of the invention is the use of the composition for manufacturing a film, a coating on a support, a hollow fiber, an artificial leather, a polymeric fiber, a membrane, a separator or an electrode for batteries, an electronic circuit or a sheath for the protection of electric cables.

Another subject matter of the invention is a compound of formula:

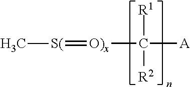

in which:
x is equal to 1 or 2;
R¹ and R² represent, independently of one another, a hydrogen atom or an alkyl group comprising from 1 to 6 carbon atoms;
A represents one of the following groups: A1, A2, A3

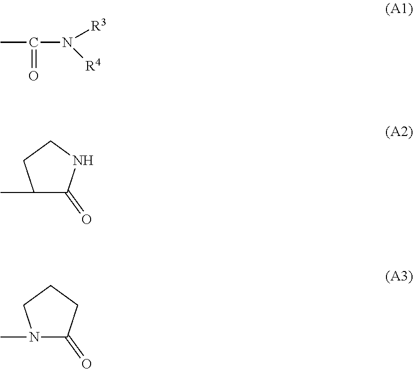

and, when A=A1, then:
n ranges from 2 to 10, if x=1;
n ranges from 3 to 10, if x=2;
R³ and R⁴ represent, independently of one another, a hydrogen atom or an alkyl group comprising from 1 to 20 carbon atoms;
   and, when A=A2 or A3, then n is equal to 1 or 2.

In one embodiment of the invention, a subject matter of the latter is preferably a compound of formula:

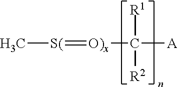

in which:
x is equal to 1 or 2;
R¹ and R² represent, independently of one another, a hydrogen atom or an alkyl group comprising from 1 to 6 carbon atoms;
A represents one of the following groups: A1, A2, A3

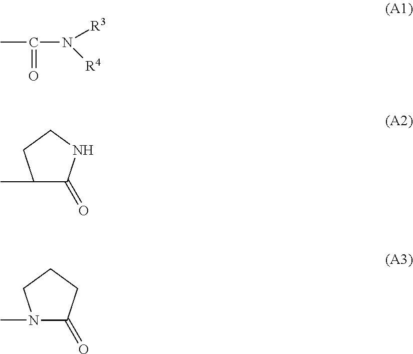

and, when A=A1, then:

n ranges from 2 to 10 if x=1 and R³ and R⁴ each represent a hydrogen atom, or one only of the R³ and R⁴ groups represents a hydrogen atom and the other group represents an alkyl group comprising from 1 to 20 carbon atoms;

n ranges from 3 to 10 if x=2 and R³ and R⁴ are chosen from the group consisting of the methyl and ethyl groups;

and, when A=A2 or A3, then n is equal to 1 or 2.

According to a further embodiment:
R¹ and R² each represent the hydrogen atom;
R³ and R⁴ represent, independently of one another, an alkyl group comprising from 1 to 20 carbon atoms.

According to one embodiment, R³ and R⁴ are chosen from the group consisting of the methyl and ethyl groups.

According to one embodiment, R³ and R⁴ each represent the methyl group.

### DESCRIPTION OF EMBODIMENTS OF THE INVENTION

The invention is now described in greater detail and in a nonlimiting manner in the description which follows.

To this end, the invention provides for the use, as solvent, of a compound comprising:
at least one sulfoxide functional group and/or one sulfone functional group, and
at least one amide functional group,
   in order to dissolve a chemical entity.

In a preferred embodiment, the compound corresponds to the following formula (I):

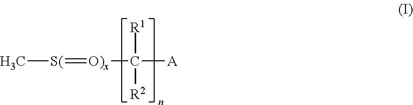

in which:
x is equal to 1 or 2;
R¹ and R² represent, independently of one another, a hydrogen atom or an alkyl group comprising from 1 to 6 carbon atoms;
n ranges from 1 to 10, preferably from 1 to 6 and more preferably from 1 to 2;
A represents one of the following groups:

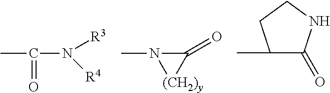

where:

R³ and R⁴ represent, independently of one another, a hydrogen atom or an alkyl group comprising from 1 to 20 carbon atoms;
y ranges from 1 to 10, preferably from 2 to 4 and is preferably equal to 3.

The value x=1 indicates the presence, in the molecule, of a sulfoxide —S(═O)— group. The value x=2 indicates the presence of a sulfone —S(═O)₂— group.

In one embodiment, neither R³ nor R⁴ is a hydrogen atom. For example, R³ and R⁴ are chosen from the group consisting of the methyl and ethyl groups. In this case, R³ and R⁴ preferably each represent the methyl group.

In another embodiment, one only of R³ and R⁴ represents a hydrogen atom.

R¹ and R² preferably each represent a hydrogen atom.

Examples of preferred compounds comprising an acyclic amide functional group are:
CH₃—S(═O)—CH₂—C(═O)—N(CH₃)(CH₃)
CH₃—S(═O)—(CH₂)₂—C(═O)—N(CH₃)(CH₃)
CH₃—S(═O)₂—CH₂—C(═O)—N(CH₃)(CH₃)
CH₃—S(═O)₂—(CH₂)₂—C(═O)—N(CH₃)(CH₃)

Examples of preferred compounds comprising a cyclic amide functional group are:

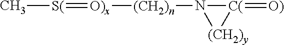

with, preferably, n=1 or 2 and y=3;

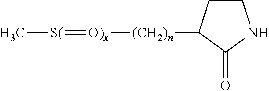

with x=1 or 2 and n ranging from 1 to 10.

Among the compounds comprising a cyclic amide functional group, examples of preferred compounds are:

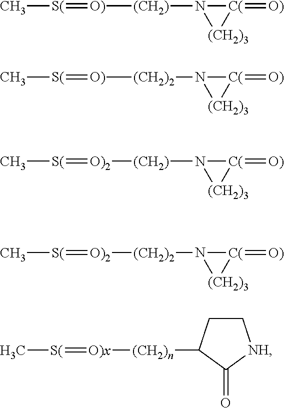

with x=1 and n=2.

It is possible to prepare and use a composition consisting of a mixture of at least two compounds according to the invention.

According to a preferred embodiment, this composition does not contain compounds acting as solvent, other than the compounds of the invention.

Although the invention makes it possible to use the compound according to the invention without other solvent, it is possible to envisage using it as a mixture with other solvents, for example dimethyl sulfoxide (DMSO).

Other solvents (cosolvents) can be used in combination with the solvent according to the invention and optionally DMSO, as indicated above. These cosolvents can be chosen from:
water;
ketones, such as acetone, methyl ethyl ketone (MEK), methyl isobutyl ketone (MIBK), hexanone, cyclohexanone, ethylamine ketone, isophorone, trimethylcyclohexanone, γ-butyrolactone or diacetone alcohol;
amines, such as monoethanolamine (MEoA), diethanolamine (DEoA), propanolamine (PoA), butylisopropanolamine (BiPoA), isopropanolamine (iPoA), 2-[2-(3-aminopropoxy)ethoxy]ethanol, N-(2-hydroxyethyl)diethylenetriamine, (3-methoxy)propylamine (MoPA), 3-isopropoxypropylamine (IPOPA), monoethylamine, diethylamine, diethylaminopropylamine (DEAPA), triethylamine (TEA) or acetonitrile;
alcohols, such as ethanol, methanol, propanol, isopropanol, glycerol, diacetone alcohol, butanol, methyl isobutyl carbinol, hexylene glycol or benzyl alcohol;
ethers, such as tetrahydrofuran (THF), methylfuran, methyltetrahydrofuran, tetrahydropyran or glycol dialkyl ether;
esters, such as dibasic esters, dimethyl glutarate, dimethyl succinate, dimethyl adipate, butyl acetate, ethyl acetate, diethyl carbonate, dimethyl carbonate, propylene carbonate, ethyl methyl carbonate, glycerol carbonate, dimethyl 2-methylglutarate, dimethyl 2-methyladipate, dimethyl 2-methylsuccinate, n-butyl propionate, benzyl acetate or ethyl ethoxypropionate;
sulfones, such as dimethyl sulfone or sulfolane;
aromatic compounds, such as toluene and xylene;
acetals, such as methylal, ethylal, butylal, dioxolane and 2,5,7,10-tetraoxaundecane (TOU);
glycol ethers of E or P type, such as dipropylene glycol dimethyl ether (DPGDME) or dipropylene glycol methyl ether.

Mention may also be made, as examples of other solvents, of: N-butylpyrrolidone, N-isobutylpyrrolidone, N-(t-butyl)pyrrolidone, N-(n-pentyl)pyrrolidone, N-((methyl-substituted)butyl)pyrrolidone, N-propyl- or N-butylpyrrolidone, the ring of which is methyl-substituted, or N-(methoxypropyl)pyrrolidone, polyglyme, ethyl diglyme, 1,3-dioxolane or methyl 5-(dimethylamino)-2-methyl-5-oxopentanoate.

The amount of other solvent, such as DMSO, can then range up to 90% by weight of the mixture. Generally, the compound according to the invention is predominant with respect to the other solvent. “Predominant” is understood to mean more than 50% by weight, with respect to the total weight of the mixture.

The compound according to the invention can be used as replacement for solvents such as DMF, NMP or DMAc for preparing, in the form of solutions, chemicals and materials, for formulating chemical compositions or for treating surfaces. It makes it possible to more easily implement the dissolution processes in comparison with the processes of the prior art as the dissolution involves the use of just one solvent instead of two or more solvents. Furthermore, the process of recycling the solvents is simplified because just one solvent is recycled.

The compound according to the invention can be used to dissolve polymers, chemical entities, in particular chemical entities active in the pharmaceutical or plant protection field, this list of products to be dissolved not being limiting.

Plant protection compositions can be obtained in the form of emulsifiable concentrates intended to be diluted in water by a farmer, before application on a field. A plant protection composition can comprise:
an active chemical entity,
the compound according to the invention,
one or more surfactants,
optionally water.

The plant protection compositions include herbicides, insecticides and fungicides.

The compound according to the invention can also be used in the preparation of pharmaceutical compositions.

The compound according to the invention can be used as solvent for dissolving a polymer or can act as plasticizing agent in a thermoplastic polymer composition. The polymer is preferably a polymer which can be treated in the molten state, that is is say that it can be given different shapes, such as films, fibers or tubes, using conventional melt extrusion or injection molding techniques or by casting. The weight of the polymer can represent from 5% to 30% of the weight of the mixture consisting of the solvent and the polymer.

The polymer can be chosen from the group consisting of polyvinylidene fluoride (PVDF), polytetrafluoroethylene (PTFE), a polyurethane (PU), a polyethersulfone (PES), a polysulfone (PSU), a polyphenylsulfone (PPSU), a polyimide (PI), a polyester-imide (PEI) and a polyamide-imide (PAI). In a preferred embodiment, the polymer is a fluoropolymer, such as polyvinylidene fluoride (PVDF) or polytetrafluoroethylene (PTFE).

It is possible to envisage dissolving a blend of several polymers. Mention may be made of the following examples of blends:
a polyimide with a sulfonated polymer,
a polyamide-imide (PAI) with an aromatic sulfonated polymer (PSU), or
a polyamide-imide with a polyethersulfone (PES).

In order to dissolve the polymer, the compound of the invention is mixed with the polymer and the mixture is heated with stirring at a temperature generally of less than 180° C., for example of less than 100° C., in order to obtain a solution exhibiting just one homogeneous and transparent phase.

The compound can advantageously be used as solvent in a process for the preparation of sulfonated membranes. A sulfonated polymer solution is prepared by mixing the compound with the sulfonated polymer. When a homogeneous and transparent solution is obtained, the solution is shaped into a film by evaporation of the solvent (dry process). The film obtained can be flat if the solution is left to dry on a flat support. It can also have a tubular shape if it is deposited around a support material having a tubular shape. Another possible shaping is the hollow fiber shape obtained by spinning the polymer solution, followed by dipping in a third solvent (for example water), making it possible to precipitate the polymer and to cause the solvent to migrate from the polymer solution toward the third solvent (coagulation: wet process).

The compound can also advantageously be used in the manufacture of a battery electrode. This is because fluoropolymers, such as PVDF and PTFE, are conventionally used as plasticizers in conventional processes for the manufacture of electrodes. Their role is to improve the adhesion of an active material to a current collector on which it is deposited. The PVDF or the PTFE is generally mixed with the active material and the mixture is transformed into a paste by the addition of a solvent, such as N-methylpyrrolidone. In point of fact, as explained above, N-methylpyrrolidone is regarded as toxic. The invention makes it possible to avoid having recourse to N-methylpyrrolidone.

The compound according to the invention can also advantageously be used to manufacture a membrane or a coating for a substrate. The substrate can consist of plastic, of metal or of glass. The coating can have a flat shape (film) or a tubular shape (sheath). It can be a plastic sheath coating an electric cable. The membrane or the coating is obtained by depositing a solution containing the polymer on one or more surfaces of the substrate and by allowing the solvent to evaporate, optionally while heating in order to accelerate the evaporation.

The compound can be used to create a coating on the surface of a battery separator. Such a separator generally consists of porous polyolefin but can also consist of PTFE, of polystyrene or of polyethylene terephthalate (PET). A solution containing the polymer is applied to one or both faces of the separator and then the solvent is allowed to evaporate. After drying, a porous polymer coating is obtained on the surface of the separator.

The compound can be used to manufacture polyurethane-based artificial leather according to a process well known to a person skilled in the art. Such an artificial leather can be obtained by production of a film by a process of coating of the PU polymer solution, followed by drying (heat treatment: dry process) in order to evaporate the solvent. Such an artificial leather can also be obtained by production of a film by a process of impregnation on a support of the polymer solution, followed by dipping in a third solvent (for example water), making it possible to precipitate the polymer and to cause the solvent to migrate from the polymer solution toward the third solvent (coagulation: wet process).

The compound can also be used as solvent in a cleaning, degreasing or stripping composition, for example for stripping paint. It dissolves the product to be removed, thus producing a degreasing or stripping action. It can be used as cleaning solvent on hard surfaces, such as floors or furniture surfaces. It can be used to degrease manufactured products. It can be used on textile surfaces.

The composition can also be used in a lubricant composition, in a coating composition, for example in a paint composition, in a pigment or ink composition, or in the form of dispersions, of suspensions and others. It can be used as coalescence agent in a paint composition.

The compounds of the invention can be synthesized in a way known to a person skilled in the art by reacting a first molecule, comprising a sulfone and/or sulfoxide functional group, with reactive groups of a second molecule, comprising an amide functional group.

### Examples of the Synthesis of Compounds which can be Used as Solvent

(1) The compound of formula CH₃—S(═O)—(CH₂)—C(═O)—N(CH₃)₂ can be obtained from CH₃S—CH₂—C(═O)O—CH₃ (CAS No. 16630-66-3), available from Aldrich. In a first stage, CH₃S—CH₂—C(═O)O—CH₃ is reacted with hydrogen peroxide (H₂O₂) in order to oxidize the sulfur and to form a sulfoxide functional group. The compound CH₃S(═O)—CH₂—C(═O)O—CH₃ is obtained. In a second stage, CH₃S(═O)—CH₂—C(═O)O—CH₃ is reacted with dimethylamine in order to obtain CH₃—S(═O)—CH₂—C(═O)—N(CH₃)₂.

(2) The compound of formula CH₃—S(═O)—(CH₂)₂—C(═O)—N(CH₃)₂ can be obtained from CH₂═CH—C(═O)—N(CH₃)₂ (CAS No. 2680-03-7), available from Aldrich. In a first stage, CH₂═CH—C(═O)—N(CH₃)₂ is reacted with methanethiol CH₃SH. The product of the reaction is CH₃S—(CH₂)₂—C(═O)—N(CH₃)₂. In a second stage, CH₃S—(CH₂)₂—C(═O)—N(CH₃)₂ is oxidized with hydrogen peroxide H₂O₂ in order to obtain CH₃—S(═O)—(CH₂)₂—C(═O)—N(CH₃)₂.

According to one method of synthesis, it is possible to start from an ester of a carboxylic acid comprising an ethylene functional group, such as CH₂═CH—(CH₂)ₙ—(C═O)—O—CH₃.

1) This ester is reacted with methanethiol in order to graft a sulfur atom thereto.

CH₂═CH—(CH₂)ₙ—(C═O)—O—CH₃+CH₃SH→CH₃—S—(CH₂)ₙ₊₂—(C═O)—O—CH₃

2) An amine, preferably a secondary amine H—N—(R¹)(R²), is reacted with the product obtained in stage 1):

CH₃—S—(CH₂)ₙ₊₂—(C═O)OCH₃+H—N—(R¹)(R²)→CH₃—S—(CH₂)ₙ₊₂—(C═O)N(R¹)(R²)

3) The product obtained in stage 2) is oxidized using hydrogen peroxide or another oxidant:

CH₃—S—(CH₂)ₙ₊₂—(C═O)N(R¹)(R²)+H₂O₂→CH₃—S(═O)—(CH₂)ₙ₊₂—(C═O)N(R¹)(R²)

4) It is optionally possible to continue the oxidation by oxidizing the sulfoxide —S(═O) functional group to give a sulfone —S(═O)₂ functional group.

In an alternative form, stages 2 and 3 can be reversed. It is also possible to start from a carboxylic acid comprising an ethylene functional group, such as CH₂═CH—(CH₂)ₙ—(C═O)—OH.

According to another method of synthesis, it is possible to start from acid chloride derivatives, such as CH₂ClCOOH, in order to react them with NaSCH₃ and then H₂SO₄ in order to obtain CH₃SCH₂COOH, which can subsequently react with H₂O₂ to give CH₃SOCH₂COOH. The latter compound can react with (CH₃)₂NH in order to obtain CH₃—S(═O)—CH₂—C(═O)—N(CH₃)₂.

According to another method of synthesis, it is possible to start from acid chloride ester derivatives, such as CH₂ClCOOCH3, in order to react them with CH₃SH in order to obtain CH₃SCH₂COOCH₃, which can subsequently react with H₂O₂ to give CH₃SOCH₂COOCH₃. The latter compound can react with (CH₃)₂NH in order to obtain CH₃—S(═O)—CH₂—C(═O)—N(CH₃)₂.

According to another method of synthesis, it is possible to start from dimethyl sulfoxide, DMSO, and to react it with sodium hydride, NaH, in order to obtain the dimsyl ion (CH₃—S(═O)—CH₂⁻Na⁺) and H₂. The dimsyl ion can react with ClCH₂—C(═O)—N(CH₃)₂ in order to obtain CH₃—S(═O)CH₂CH₂C(═O)—N(CH₃)₂+NaCl.

The compound of formula:

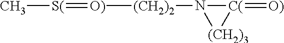

can be obtained by reacting 2-vinylpyrrolidone of formula:

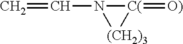

with methanethiol, followed by an oxidation with H₂O₂, for example.

1-7. (canceled)

8. A composition comprising:
a compound a) comprising:
at least one sulfoxide functional group and/or one sulfone functional group, and
at least one amide functional group, and
at least one chemical entity b) dissolved in the compound a).

9. The composition as claimed in claim 8, in which the compound a) is present at at least 45% by weight in the composition, the limits being included.

10. The composition as claimed in claim 8, in which the chemical entity is chosen from a polymer or a chemical entity active in the pharmaceutical or plant protection field.

11. The composition as claimed in claim 10, in which b) is chosen from the group consisting of polyvinylidene fluoride (PVDF), polytetrafluoroethylene (PTFE), a polyurethane (PU), a polyimide (PI), a polyester-imide (PEI), a polyamide-imide (PAI) and sulfonated polymers.

12. The composition as claimed in claim 8, which is suitable as a plant protection composition, pharmaceutical composition, stripping composition, degreasing composition, cleaning composition, lubricating composition, coating composition or pigment composition.

13. (canceled)

14. A compound of formula: 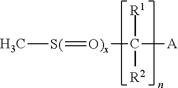
in which:
x is equal to 1 or 2;
R¹ and R² represent, independently of one another, a hydrogen atom or an alkyl group comprising from 1 to 6 carbon atoms;
A represents one of the following groups: A1, A2, A3 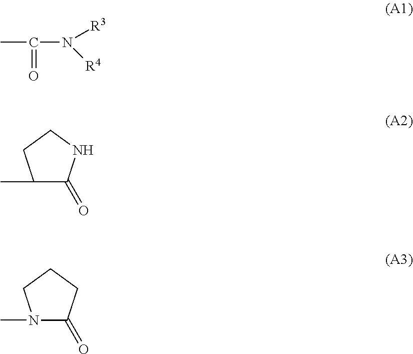
and, when A=A1, then:
n ranges from 2 to 10, if x=1;
n ranges from 3 to 10, if x=2;
R³ and R⁴ represent, independently of one another, a hydrogen atom or an alkyl group comprising from 1 to 20 carbon atoms;
and, when A=A2 or A3, then n is equal to 1 or 2.

15. The compound as claimed in claim 14, in which R³ and R⁴ are chosen from the group consisting of the methyl and ethyl groups.

16. The compound as claimed in claim 14, wherein R³ and R⁴ each represent a methyl group.

17. The compound as claimed in claim 14, wherein R¹ and R² each represent a hydrogen atom.

18. The composition as claimed in claim 8, wherein the compound a) is represented by formula (I): 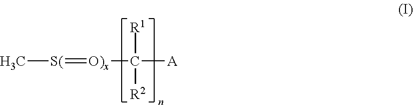
wherein
x is equal to 1 or 2;
R¹ and R² represent, independently of one another, a hydrogen atom or an alkyl group comprising from 1 to 6 carbon atoms;
n ranges from 1 to 10;
A represents one of the following groups: 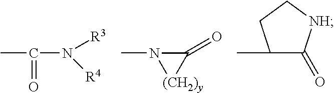
R³ and R⁴ represent, independently of one another, a hydrogen atom or an alkyl group comprising from 1 to 20 carbon atoms; and
y ranges from 1 to 10.

19. The composition as claimed in claim 18, wherein n ranges from 1 to 6.

20. The composition as claimed in claim 18, wherein n ranges from 1 to 2.

21. The composition as claimed in claim 18, wherein R¹ and R² each represent a hydrogen atom.

22. The composition as claimed in claim 18, wherein R³ represents a hydrogen atom and R⁴ represents an alkyl group comprising from 1 to 20 carbon atoms.

23. The composition as claimed in claim 18, wherein R³ and R⁴ each represent an alkyl group comprising from 1 to 20 carbon atoms.

24. The composition as claimed in claim 18, wherein the compound is chosen from the compounds of formula: 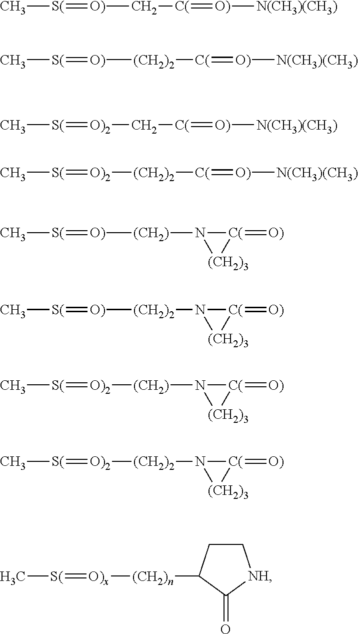
with x=1 and n=2.

25. The composition as claimed in claim 8, wherein b) is a polymer.

26. A method of preparing the composition as claimed in claim 8, comprising combining a) and b).

27. A method of manufacturing a film, a coating on a support, a hollow fiber, an artificial leather, a polymeric fiber, a membrane, a separator or an electrode for batteries, an electronic circuit or a sheath for the protection of electric cables, comprising incorporating the composition as claimed in claim 8 into a film, a coating on a support, a hollow fiber, an artificial leather, a polymeric fiber, a membrane, a separator or an electrode for batteries, an electronic circuit or a sheath for the protection of electric cables.

28. A method of manufacturing a film, a coating on a support, a hollow fiber, an artificial leather, a polymeric fiber, a membrane, a separator or an electrode for batteries, an electronic circuit or a sheath for the protection of electric cables, comprising incorporating the compound as claimed in claim 14 into a film, a coating on a support, a hollow fiber, an artificial leather, a polymeric fiber, a membrane, a separator or an electrode for batteries, an electronic circuit or a sheath for the protection of electric cables.

29. A process for dissolving a chemical species comprising contacting the chemical species with an effective amount of a compound comprising:
at least one sulfoxide functional group and/or one sulfone functional group, and
at least one amide functional group.

30. The process as claimed in claim 29, wherein the chemical species is a polymer.

31. The process as claimed in claim 29, wherein the compound is represented by formula (I): 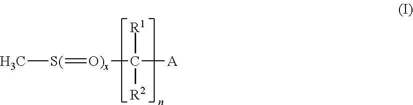
wherein
x is equal to 1 or 2;
R¹ and R² represent, independently of one another, a hydrogen atom or an alkyl group comprising from 1 to 6 carbon atoms;
n ranges from 1 to 10;
A represents one of the following groups: 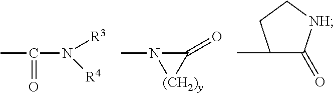
R³ and R⁴ represent, independently of one another, a hydrogen atom or an alkyl group comprising from 1 to 20 carbon atoms; and
y ranges from 1 to 10.

32. The process as claimed in claim 31, wherein n ranges from 1 to 6.

33. The process as claimed in claim 31, wherein n ranges from 1 to 2.

34. The process as claimed in claim 31, wherein R¹ and R² each represent a hydrogen atom.

35. The process as claimed in claim 31, wherein R³ represents a hydrogen atom and R⁴ represents an alkyl group comprising from 1 to 20 carbon atoms.

36. The process as claimed in claim 31, wherein R³ and R⁴ each represent an alkyl group comprising from 1 to 20 carbon atoms.

37. The process as claimed in claim 31, wherein the compound is chosen from the compounds of formula: 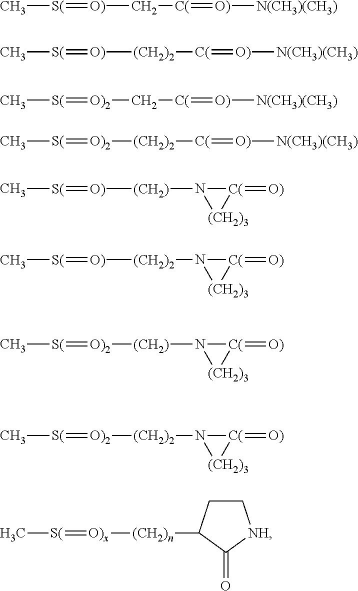
with x=1 and n=2.
